(19) United States
(12) Patent Application Publication
(10) Pub. No.: US 2007/0123970 A1
(43) Pub. Date: May 31, 2007

(54) Bifurcation stent with overlapping crimped struts

(75) Inventor: Jason T. Lenz, Maplewood, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: BOSTON SCIENTIFIC SCIMED, INC., Maple Grove, MN (US)

(21) Appl. No.: 11/289,085

(22) Filed: Nov. 29, 2005

(51) Int. Cl. A61F 2/06; B23P 11/02

(52) U.S. Cl. 623/1.11; 623/1.15; 623/1.35; 29/508

(57) ABSTRACT

A bifurcated stent has an expanded and an unexpanded state with an outer surface and an inner surface with a wall extending there between and the inner surface defining an inner stent lumen. The stent has a primary stent body with a proximal end and a distal end with a band circumferential there between. A side branch portion is disposed in the band circumferential and in the unexpanded state the band circumferential has at least one overlapped section such that the at least one overlapped section has a greater density of stent material than the rest of the stent. In the expanded state the side branch portion extends obliquely from the primary stent body.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) may affect only one of the vessels (i.e., either of the branch vessels or the parent vessel) two of the vessels, or all three vessels.

Many prior art stents however are not wholly satisfactory for use where the site of desired application of the stent is juxtaposed or extends across a bifurcation in an artery or vein such, for example, as the bifurcation in the mammalian aortic artery into the common iliac arteries.

There remains a need for innovative bifurcated stents which may be deployed using a single balloon for the main section and an alternate mechanism for the side branch support. There also remains a need for bifurcated stents with innovative designs in the region of the bifurcation to provide adequate coverage of the carina. The art referred to and/or described above is not intended to constitute an admission that any patent, publication or other information referred to herein is “prior art” with respect to this invention.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In at least one embodiment, the invention is directed to a bifurcated stent having a primary stent body with a band circumferential such that in the unexpanded state the band circumferential has at least one overlapped section with a greater density of stent material than the rest of the stent and has a side branch portion. In at least one embodiment, the band circumferential is disposed between the distal and proximal end of the primary stent body. In at least one embodiment, in the expanded state the side branch portion can extend obliquely from the primary stent body.

In at least one embodiment, in the unexpanded state at least one overlapped section can be disposed at the side branch portion.

In at least one embodiment, in the unexpanded state at least one overlapped section can be disposed opposite the side branch portion.

In at least one embodiment, the at least one overlapped section comprises struts such that in the unexpanded state a portion of the struts overlap a different portion of the struts and in the expanded state the struts extend obliquely from the primary stent body and form a side branch. In at least one embodiment, the side branch can have a side branch lumen that is in communication with the inner stent lumen of the primary stent body.

In at least one embodiment, the overlapped section can comprise two flap portions having ends which extend towards one another.

In at least one embodiment, the overlapped section can comprise two flap portions having ends which extend away from one another.

In at least one embodiment, one overlapped section can be disposed at the side branch portion and another overlapped section can be disposed opposite the side branch portion.

In at least one embodiment, the stent can be self expandable.

In at least one embodiment, the stent can be balloon expandable.

In at least one embodiment, the invention is directed to a crimper having multiple first crimping members and at least one pair of second crimping members. In at least one embodiment, the first crimping members are constructed and arranged about an aperture and define a maximum circular diameter. In at least one embodiment, the first crimping members can be moveable such that movement of the first crimping members increase or decrease the maximum circular diameter. In at least one embodiment, at least one pair of second crimping members are moveable separately from the first crimping members and capable of extending into the maximum circular diameter defined by the first crimping members.

In at least one embodiment, the first crimping members are moveable simultaneously with one another.

In at least one embodiment, a stent can be disposed within the maximum circular diameter of the aperture.

In at least one embodiment, movement of the at least one pair of second crimping members can form an overlapped portion of the stent.

In at least one embodiment, at least one second crimping member can extend from at least one of the first crimping members. In at least one embodiment, the second crimping member can be slidable within a first crimping member.

In at least one embodiment, at least one first crimping member can be separated from an adjacent first crimping member by a gap space. In at least one embodiment, a second crimping member can move within the gap space.

In at least one embodiment, the crimper can have multiple pairs of second crimping members.

In at least one embodiment, the method of crimping a stent comprises:

providing one of the stent crimpers described above;

inserting a stent into the aperture of the crimper;

acting on the stent with at least one pair of second crimping members such that the stent deforms and forms at least one flap; and

acting on the stent with multiple first members which crimp the stent such that the at least one flap overlaps other portions of the crimped stent, the multiple members further crimping the stent.

In at least one embodiment of the method the stent can be disposed about a catheter.

In at least one embodiment of the method multiple portions of the stent are acted upon by at least one pair of second crimping members such that multiple flaps are formed.

In at least one embodiment of the method the stent can have a primary inner lumen which is substantially circular before crimping, having two bulging portions and one narrow portion during crimping. In at least one embodiment the primary inner lumen returns to being substantially circular when crimped.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1a is a side view of an embodied deployed bifurcated stent.

FIG. 1b is a side view of an deployed bifurcated stent.

FIG. 2 is a cross-sectional side view of an embodied stent disposed about a catheter.

FIG. 3a is a cross-sectional end view of an embodied stent.

FIG. 3b is a cross-sectional end view of an embodied stent.

FIG. 4 is a cross-sectional side view of an embodied stent disposed about a catheter.

FIG. 5a is a view of an embodied crimper with an embodied stent disposed therein.

FIG. 5b is a view of an embodied crimper with an embodied stent disposed therein and being partially deformed.

FIG. 5c is a view of an embodied crimper with an embodied stent disposed therein.

FIG. 5d is a view of an embodied crimper with an embodied stent disposed therein having been reduced.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

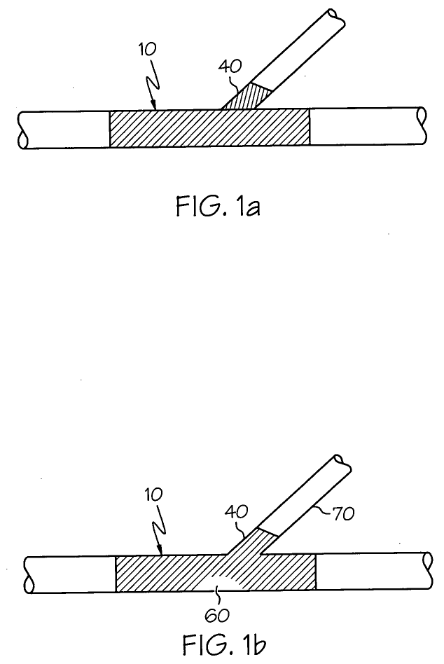

In the embodied stent 10 as shown in FIG. 1a the stent 10 provides full scaffolding after expansion of the side branch 40 into a secondary lumen as the stent was crimped with an overlapped portion which provides the extra scaffolding necessary to provide consistent scaffolding throughout the stent. The overlapped portions or sections provide extra material that is nested against a sidewall of the stent 10 in the unexpanded state. When in the expanded state the overlapped sections unfold and provide coverage and support to the vessel lumen. The overlapping is shown in more detail below.

This is not the case in some prior art bifurcated stent designs as shown in FIG. 1b. Here, the stent 10 sacrifices some scaffolding in certain areas (e.g. area 60 which is opposite the side branch 40) in order for the bifurcated spent to expand and supply scaffolding to the secondary lumen 70. This can weaken the effectiveness of the stent 10 as the stent has lessened contact with the vessel wall.

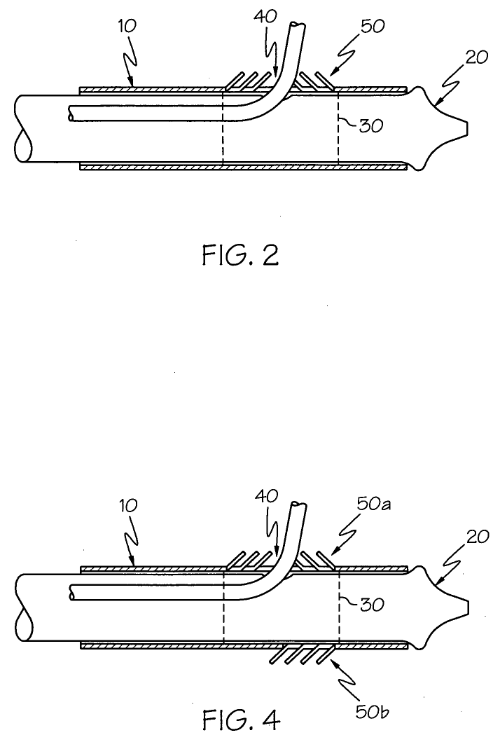

In FIG. 2 an embodied stent 10 disposed about a catheter 20 is shown. A portion of the stent 10 is designated as the band circumferential 30 and is a circumferential region in which a side branch 40 and/or overlapping/nested portion 50 may be disposed. The side branch 40 can include overlapped or nested portions. The overlapped portions 50 allow for higher metal coverage on the deployed stent 10.

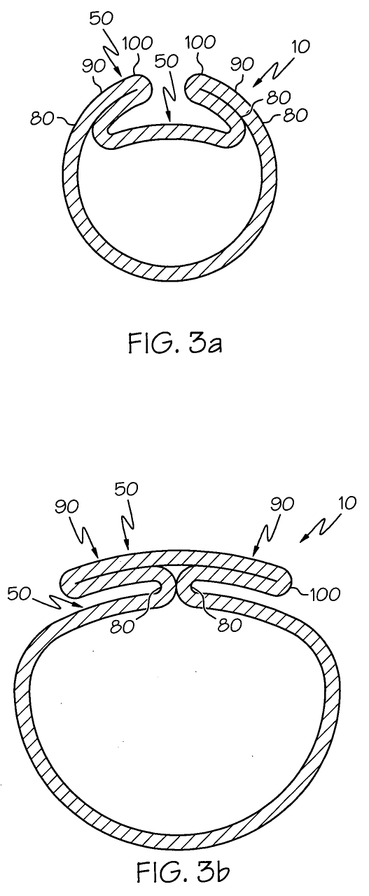

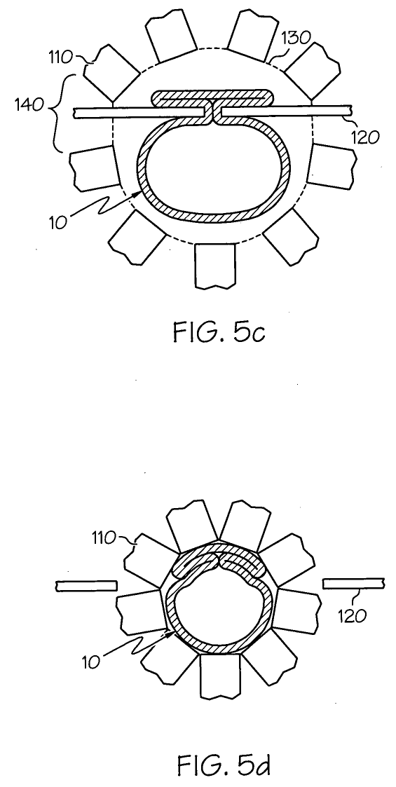

In FIG. 3a an overlapped portion 50 of stent 10 is shown in cross-section. The overlapped portion 50 can be formed by crimping first crimp portions 80 of the stent 10 to form flaps 90 having ends 100. Here, the ends 100 of the flaps 90 extend toward one another. In some embodiments multiple flaps 90 are formed. The flaps 90 can be formed by multiple sets of second crimping members 120 (shown in FIGS. 5a-5d) or by a single set 120 by rotating the stent 10.

In FIG. 3b the stent 10 has multiple flaps 90 with a single pair of first crimp portions 80. Here the ends 100 extend away from one another. The extra scaffolding material upon expansion can then provide full coverage about the stent 10.

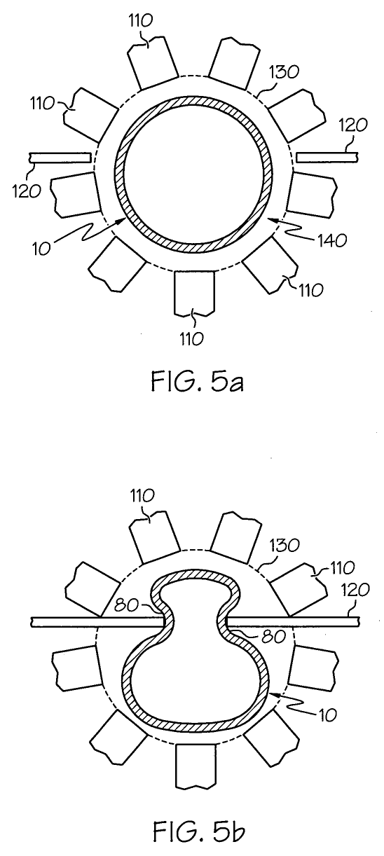

The stent 10 can have multiple overlapped portions 50 as shown in FIG. 4. Overlapped portion 50a is located at side branch 40. Overlapped portion 50b is located opposite side branch 40. Increased scaffolding in these two locations may reduce the restenosis that occurs in these problematic areas of a vessel lumen.

The stent 10 can be crimped in a crimper having first crimping members 110 and second crimping members 120 as shown in FIGS. 5a-5d. The first crimping members define a maximum circular diameter 130 which changes in size as the first crimping members 110 are moved in or out from the center of the aperture 140. The maximum circular diameter can be defined as the largest diameter that can fit within the aperture between the first crimping members 110 at their given position. In some embodiments, all the first crimping members move simultaneously.

The second crimping members 120 can move independently of the first crimping members and may move into the maximum circular diameter 130. In some embodiments as shown in FIG. 5b, the second crimping members can contact the stent 10 at crimp portions 80. As shown here, continued crimping at this position 80 can result in flaps being formed as shown in FIG. 5c. The second crimping members can then be moved out of contact with the stent and first crimping members can move inward to further reduce the entire stent 10 as shown in FIG. 5d. In some embodiments, the second crimping members 120 move within gap space 140. In some embodiments, a second crimping member 120 extends from one of the first crimping members 110 and is moveable within a space or lumen within the first crimping member 110. It should be noted that in some embodiments a single second crimping member can be used such that when extended the second crimping member contacts the stent to form a flapped portion. It should be further noted that after the flaps are formed as shown in FIG. 5c, the stent may then be placed into another and/or more standard crimper in order to perform the final crimping of the flaps over the overlapped portions.

Before being crimped or reduced in any way, the inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto. The therapeutic agent can be applied in a variety of ways and can include therapeutic agent being applied in some locations more than others.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims may be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

What is claimed is:

1. A bifurcated stent having an expanded and an unexpanded state, the stent comprising:
an outer surface and an inner surface with a wall extending therebetween, the inner surface defining an inner stent lumen,
a primary stent body having a proximal end and a distal end with a band circumferential therebetween, a side branch portion disposed in the band circumferential, in the unexpanded state the band circumferential has at least one overlapped section, the at least one overlapped section having a greater density of stent material than at least one adjacent section of the stent, in the expanded state the side branch portion extending obliquely from the primary stent body.

2. The bifurcated stent of claim 1 wherein in the unexpanded state the at least one section is disposed at the side branch portion.

3. The bifurcated stent of claim 1 wherein in the unexpanded state the at least one section is disposed opposite the side branch portion.

4. The bifurcated stent of claim 2 wherein the at least one section comprises struts, in the unexpanded state a portion of the struts overlap a different portion of the struts, in the expanded state the struts extend obliquely from the primary stent body and form a side branch, the side branch having a side branch lumen that is in communication with the inner stent lumen of the primary stent body.

5. The bifurcated stent of claim 1 wherein the at least one overlapped section comprises two flap portions having ends, the ends extending towards one another.

6. The bifurcated stent of claim 1 wherein the at least one overlapped section comprises two flap portions having ends, the ends extending away from one another.

7. The bifurcated stent of claim 1 wherein one overlapped section is disposed at the side branch portion and another overlapped section is disposed opposite the side branch portion.

8. The bifurcated stent of claim 1 wherein the stent is self expandable.

9. The bifurcated stent of claim 1 wherein the stent is balloon expandable.

10. A crimper comprising:
multiple first crimping members and at least one pair of second crimping members, the first crimping members constructed and arranged about an aperture and defining a maximum circular diameter, the first crimping members moveable such that movement of the first crimping members increase or decrease the maximum circular diameter;
the at least one pair of second crimping members moveable separately from the first crimping members.

11. The crimper of claim 10 wherein the at least one pair of second crimping members are capable of extending into the maximum circular diameter defined by the first crimping members.

12. The crimper of claim 10 wherein the first crimping members are moveable simultaneously with one another.

13. The crimper of claim 10 further comprising a stent disposed within the maximum circular diameter of the aperture.

14. The crimper of claim 11 wherein movement of the at least one pair of second crimping members forms an overlapped portion of the stent.

15. The crimper of claim 10 wherein at least one second crimping member extends from at least one of the first crimping members, the second crimping member slidable within the at least one of the first crimping members.

16. The crimper of claim 10 wherein at least one first crimping member is separated from an adjacent first crimping member by a gap space, a second crimping member moveable within the gap space.

17. The crimper of claim 10 having multiple pairs of second crimping members.

18. A method of crimping a stent comprising:
(a) providing a stent crimper having multiple first crimping members and at least one pair of second crimping members, the first crimping members constructed and arranged about an aperture and defining a maximum circular diameter, the first crimping members moveable such that movement of the first crimping members increase or decrease the maximum circular diameter, the at least one pair of second crimping members moveable separately from the first crimping members;
(b) inserting a stent into the aperture of the crimper;
(c) acting on the stent with the at least one pair of second crimping members such that the stent deforms and forms at least one flap; and
(d) acting on the stent to further crimp the stent such that the at least one flap overlaps other portions of the crimped stent.

19. The method of claim 18 wherein the stent is disposed about a catheter.

20. The method of claim 18 wherein multiple portions of the stent are acted upon by at least one pair of second crimping members such that multiple flaps are formed.

21. The method of claim 18 wherein the stent has a primary inner lumen, the primary inner lumen being substantially circular before crimping, during crimping the primary inner lumen having two bulging portions and one narrow portion before returning to a substantially circular shape.

22. The method of claim 18 wherein the multiple first crimping members further crimp the stent such that the at least one flap overlaps other portions of the crimped stent.

23. The method of claim 18 wherein further crimping the stent such that the at least one flap overlaps other portions of the crimped stent is performed in another crimper.
